(19) United States
(12) Patent Application Publication
(10) Pub. No.: US 2022/0168104 A1
(43) Pub. Date: Jun. 2, 2022

(54) IMPLANT MADE OF CARRIER MATERIAL INTERSPERSED WITH BIOLOGICALLY ACTIVE DONOR MATERIAL, AND METHOD FOR PRODUCING SUCH AN IMPLANT

(71) Applicant: Karl Leibinger Medizintechnik GmbH & Co. KG, Mühlheim (DE)

(72) Inventors: Adem AKSU, Villingen--Schwenningen (DE); Frank REINAUER, Emmingen-Liptingen (DE); Tobias WOLFRAM, Dreieich (DE)

(21) Appl. No.: 17/598,529

(22) PCT Filed: Jan. 29, 2020
(86) PCT No.: PCT/EP2020/052114

§ 371 Date: Sep. 27, 2021

(30) Foreign Application Priority Data
Mar. 29, 2019 (DE) 10 2019 108 190.4

(51) Int. Cl. A61F 2/28; A61F 2/30

(52) U.S. Cl. CPC A61F 2/2875; A61F 2/3094; A61F 2/30771; A61F 2002/30062; A61F 2002/30968; A61F 2002/30677; A61F 2002/30317; A61F 2002/30242; A61F 2002/30784

(57) ABSTRACT

The invention relates to an implant (1) for introducing into a patient, having an implant body that is at least partially resorbable and is porous at least in some regions and that is made of a ceramic carrier material (2), the carrier material being provided with a donor material (3) that delivers ions to influence the patient's cellular metabolism in the implanted state, the carrier material (2) being interspersed with the donor material (3). The invention also relates to a method for producing an implant (1) of said type.

The invention relates to an implant, for example a cranial implant, for insertion into a (human) patient's body. Furthermore, the invention relates to a method for manufacturing such an implant.

In this context, an implant is a medical device that is inserted into a human or animal body, is foreign to the body and usually remains in the body for a defined period of time. Cranial implants are skull implants, i.e. implants that are used in regions of a human or animal skull.

Resorbable/bioresorbable components/materials are materials/substances that a (patient's) body can biologically absorb.

Cellular metabolism or metabolism comprises all physical and chemical processes for converting chemical starting materials into intermediate and end products in the body.

The ceramic or non-ceramic bone regeneration products as components of implants currently available or known on the market come in the form of granules, curable cements or prefabricated molded bodies with simple standard geometry. Hardly any patient-specific implants with individually three-dimensionally adapted shape, structuring and bioactive design are provided.

Materials with biological activity or bioactive substances are interactive substances that cause a positive cellular reaction and/or ‘repair’ body tissue.

It is known to coat implants with biological activation (‘coating’), wherein the coating has stability problems with respect to the implant and is unsuitable for long-term activities.

Furthermore, ceramic materials are known which are inserted directly into the patient's body as granules or viscous paste. Such implants do not allow structure-specific, geometric, pre-implant shaping. Such implants with pores distributed in a gradient-like manner can only be created by randomly changing the implant composition.

For example, EP 0 923 953 B1 discloses a medical device having at least a proportion that is implantable into a patient's body. In this regard, at least a part of the device proportion is covered with a coating for releasing at least one biologically active material, wherein the coating comprises an underlayer having an outer surface and comprises a polymeric material containing an amount of biologically active material therein for timed release therefrom. The coating further comprises a discontinuous top layer covering less than the entire outer surface of the underlayer, wherein covered and uncovered regions are formed through the entire outer surface of the underlayers. The top layer comprises a polymeric material that is free of pores and pore-formers.

Furthermore, U.S. Pat. No. 7,101,394 B2 discloses a medical device that delivers biologically active material to a patient's body. A first top layer comprises a biologically active material and optionally comprises a polymeric material arranged on the surface of the medical device. A second top layer comprising magnetic particles and a polymeric material is arranged on the first top layer. The second top layer, which has substantially no biologically active material, protects the biologically active material.

Furthermore, US 2010/145469 A1 relates to a porous implant which uses as a carrier material a bioceramic made of a biocompatible ceramic matrix and uses as a donor material a bioactive substance which can release ions. In one embodiment, the bioactive substance permeates the carrier material.

U.S. Pat. No. 5,876,446 A discloses a porous implant made of a biodegradable carrier material in which bioactive substances are enclosed which, when inserted into a patient's body, are transferred into the latter and thereby promote the ingrowth of cells.

US 2004/258732 A1 discloses an implant having a porous portion in which a bioactive bioceramic powder is uniformly distributed in a biodegradable and bioabsorbable polymer. The bioceramic is dispersed in the solubilized polymer to produce the implant.

EP 3 115 025 A1 discloses an implant having a surface layer covering a porous and biodegradable implant portion bounded on its side opposite the surface layer by a membrane layer composed of collagen.

Against this background, it is the object of the present invention to reduce or prevent the problems of the prior art and, in particular, to provide robust or stable implants that allow better ingrowth of body tissue than conventional implants.

The invention solves this object in an implant in particular in that the implant has an at least partially resorbable and at least in partial regions porous implant body made of a ceramic carrier material, for example α-TCP, β-TCP, hydroxylapatite, biphasic calcium phosphate, bioglass, β-SiAlON or bioresorbable photopolymers. In this regard, according to the invention, the carrier material is provided with a donor material that, in the implanted state, emits ions for influencing the patient's cellular metabolism, and the donor material intersperses the carrier material. Further according to the invention, it is provided that the biologically active donor material releasing ions is structurally provided in an intrinsic manner in the implant and is not provided in the form of an implant coating. Furthermore, it is provided according to the invention that the implant comprises first layers, last layers and middle layers which are surrounded by the first and last layers, wherein the first, middle and last layers have different densities/porosities.

Intrinsically intended biological activity means that the biological activity is a property of the implant itself and is not just applied to the implant from the outside. This means that the donor material is present throughout the entire implant volume. Such an implant according to the invention allows optimal ingrowth of soft body tissue and new bone formation. At the same time, the ingrowth increases the strength of the implant. In addition, the implant according to the invention is biologically active for longer than, for example, coated implants, since the biological activity of the implant according to the invention comes from the inside (is intrinsic), unlike as in coated implants, in which only the surface is biologically active.

Advantageous embodiments are the subject matter of the dependent claims and are explained in detail below.

It is conceivable that the donor material comprises ceramic particles and/or metallic particles. It is provided that the ceramic particles are bioresorbable. Such materials are particularly suitable for releasing ions and are thus resorbable and bioactive.

Furthermore, it is practical that the implant body is divided into layers or into partial regions of different density and/or porosity. Thus, the biological activity is controlled by the layer geometry of the implant as well as by the ions released by the donor material. In addition, such an implant is particularly well suited for ingrowth of body tissue.

It is also advantageous if individual pores in the implant body are connected to each other via connection channels. Such connection channels connect pores with each other so that they allow a substance exchange between the pores or across the pores and thus enable improved and longer-lasting ion release.

It is also conceivable that the donor material is arranged and concentrated in the carrier material in such a way that, when ions are released in the implanted state, the connection channels necessarily result (secondary connection channels), or that the connection channels are already present in the implant body before insertion into the patient (primary connection channels). The implanted state is a state in which the implant is inserted into a patient's body or is present in the patient's body.

It is preferred if the implant body has a total porosity between 3% and 60%, in particular between 5% and 10%, preferably between 25% and 30%, further preferably between 50% and 60%, and particularly preferably between 75% and 80%. A total porosity in this range is particularly advantageous for ingrowth of the implant.

Furthermore, it is advantageous if the pore size of the pores in the implant body lies in a range of 300 μm to 1,500 μm, in particular 350 μm to 450 μm, 800 μm to 900 μm, 1,000 μm to 1,200 μm. The pore sizes are determined in advance by planning and are then precisely implemented in terms of construction. The pore sizes are therefore not generated randomly. The selection of the pore size suitable for the respective application allows the greatest possible open pore size with simultaneously optimized mechanical conditions and enables optimum ingrowth of soft tissue and allows new bone formation in the patient's body.

Furthermore, pore gradients of 200 μm to 900 μm or up to 2500 μm are conceivable, wherein the pore gradients are each stepped by 100 μm relative to each other. It is also conceivable that the implant has a closed structure with pore gradients of more than 10 μm.

It is also advantageous if the ceramic carrier material (in the unfinished implant) is provided in the form of powder or granular ceramic particles. The granular form influences the geometric and biological properties in the implant.

It is also possible for the ceramic particles to be arranged in a partially crystalline or crystalline form. This makes it possible to achieve a more durable and long-lasting implant.

A particular configuration example is characterized in that the ceramic particles and the metal particles are spherical (ball-shaped) with a respective particle size between 5-10 μm for the metal particles and between 25-120 μm for the ceramic particles, and/or the ceramic and metal particles are cubic (cube-shaped) with an edge length between 5-25 μm for the metal particles and between 40-60 μm for the ceramic particles. In particular, a mixture of spherical and cubic particles ultimately achieves advantageous biomechanical strength.

Furthermore, it is conceivable that the implant comprises first layers (e.g. the outermost 0 to 30 layers of the implant), last layers and middle layers (main layers), which are surrounded by the first and last layers, wherein the first layers are solid, the middle layers are porous and the last layers are solid, or the first layers are porous, the middle layers are solid and the last layers are porous.

It is also practical if the implant is hydrophobic or hydrophilic on different surfaces, in particular on surfaces that are opposite to each other. This enables different possibilities for the implant to interact mechanically and physically with the body tissue of a patient. These tissue interactions can be influenced (increased or decreased) via the partial resorbability of the defined structure or geometry of the implant.

Furthermore, it may be provided that the implant is manufactured using an additive/generative manufacturing process. An implant manufactured by this method is particularly cost-effective to produce.

Furthermore, the object underlying the invention is solved by a method for manufacturing the implant according to the invention. The method comprises the following steps, which are advantageously carried out in succession and preferably in this order:

a) mixing of carrier material and donor material, which are either powdery, granular, liquid or viscous, to form a raw mixture,

b) spatially-resolved bonding (i.e. bonding of the raw mixture elements at defined locations in order to obtain a specific implant shape of the implant body) of the raw mixture, e.g. by laser sintering, into a plurality of individual layers (preferably under gradual energy input which varies depending on the individual layer),

c) superimposing and layer-by-layer bonding of the plurality of individual layers to form the completed/finished implant body.

An implant manufactured according to these steps has the advantages defined above.

For example, by producing the first layers under high energy input, the middle layers with low energy input, and the last layers with high energy input, in this example the middle of the implant, i.e. the middle layers of the implant, have more pores than the first and last layers, so that they can resorb faster.

In other words, the invention relates to three-dimensional implants produced via generative manufacturing, wherein the implants are, for example, made of α-tricalcium phosphate (α-TCP), β-tricalcium phosphate (β-TCP) and hydroxylapatite (HA) as well as mixtures of β-TCP and HA, so-called biphasic calcium phosphates (TCP), bioglass components, as well as mixtures of α-TCP, β-TCP and HA, ZrO₂, Al₂O₃, β-SiAlON, biodegradable photopolymers, ceramic composites, and metallic particle compositions. Under energy input, the ceramic particles or a composite of ceramic powder and the organic polymer matrix or an inorganic composite of a ceramic resorbable or non-resorbable material in combination with one or more metallic particles or bioglass compositions are bonded together in a spatially-resolved manner. Through the layer-by-layer bonding and subsequent solidification, a three-dimensional implant with structurally defined macroscopic and microscopic porosity is created by superimposing and bonding many individual layers.

This ensures that the implants can be manufactured in a short time and can be adapted to the anatomical region of the patient's body. Due to the different porosities in combination with an additive/generative manufacturing process, novel shape-bound gradient geometries can be presented, which may generate specific biological activities by bioresorption.

Pore sizes of approx. 600 μm allow fast ingrowth of blood vessels, connective tissue and possibly bone tissue. Since nutrient supply to vital cells within the implant scaffold is only possible over a distance of 150-200 μm, primarily by diffusion, the formation of new blood vessels represents a decisive process with regard to successful integration of the implant. The material composition and gradient design of the porosity together with specific resorption features of the implant according to the connection optimize the supply of nutrients to biological tissues. Specific structures in the range of 300-500 μm are formed as well as larger pores in the range of 800-1,200 μm. The pores are distributed in a gradient-like manner by the construction strategy, so that gradient patterns are created which enable the greatest possible open porosity with simultaneously optimized mechanical conditions.

As a result, the implant according to the invention is either completely or at least partially resorbable. This allows optimal ingrowth of soft tissue and new bone formation. This extensive, vascular ingrowth helps to transport important cells that fight infection deep into the implant. Implants of large volume or smaller implants with an increased surface area due to construction are particularly useful.

The ingrowth of soft tissue also increases the strength of the implant and the biological activity of the implants is not controlled by growth factors but by the geometry of the implant together with the resorbable components, in particular by releasing metallic and non-metallic ions. Metabolic and cell physiological reactions (chemical and physical reactions or processes within a cell) are activated or modified for the benefit of the healing process.

Thus, the implant according to the invention does not receive a top layer/coating, but the biological activation of the implant according to the invention is intrinsically structurally present in it. This makes implants more robust during insertion and the biological activation of the implant is distributed over the time that the implant is present in the patient's body.

In other words, the invention relates to a generatively manufactured, ceramic or partially ceramic, geometrically complex implant with a three-dimensional and gradient embossed, interconnecting and/or partially interconnecting, open-pored structure. Higher strength can be achieved by a different gradual energy input (e.g. an energy input between 49, 52 to 2971, 20 mJ/cm², in particular from 80 to 110 mJ/cm², preferably from 150 to 200 mJ/cm² and particularly preferably from 260 to 290 mJ/cm²) into the respective layers. Furthermore, higher strength can be achieved by different exposure/illumination durations (between 1 to 60 seconds), exposure/illumination intensities (5 to 49, 52 mW/cm²) and waiting time per individual layer of the implant. A longer exposure time in the first and main layers leads to a higher strength of the implant.

Furthermore, the additively manufactured implant receives an increase in strength, by the different energy inputs per layer and the pore strands connected by the connection channel are exposed differently. After exposure, the ceramic implant subsequently receives heat treatment (in temperature steps with the intervals 250 to 300° C., 380 to 400° C., 450 to 470° C. and 600 to 650° C.) without closing the pores. Furthermore, the heat treatment leads to an additional increase in strength, to a (micro-) structural transformation and to changed surface properties of the implant. Different heat treatment methods (in temperature steps with the intervals 750 to 800° C., 870 to 890° C., 900 to 950° C., 950 to 1050° C., 1130 to 1170° C., 1200 to 1300° C., and 1400 to 1450° C.) can be used to achieve smooth surface properties, wherein the implant is internally porous. The implant can be made from at least one or two or three or four of the previously mentioned material components.

The resorbable proportion of the implant according to the invention may be between 0 and 100%, in particular between 20 to 30% or between 45 to 50% or between 65 to 80%. From the outside to the inside (from its outer to its inner layers), the implant can be constructed in such a way that the outer layers are resorbable to a large extent, wherein the resorbability of the layers decreases continuously or discontinuously from the outer to the inner layers.

Furthermore, the implant according to the invention may have specific structures for fixation using screws or fixation devices made of titanium, medical stainless steel, resorbable metal alloys, polymers as well as resorbable polymers. The orientation of such a specific structure is in an angular structure to the implant surface between 5 to 28° degrees, 30 to 50° degrees, 55 to 75° degrees and 80 to 85° degrees. The fixation structure should have a wall thickness between 0.5 mm to 20.0 mm.

In particular, it is conceivable that the three-dimensional implant according to the invention is provided with omissions or gaps in the event of augmentation (procedure for reconstructing autologous bone using heterologous, xenogenic, or synthetic bone replacement materials) with the implant according to the invention, in particular a dental implant. The purpose of these omissions is to be fillable with autologous bone tissue or bone fragments (the patient's own bone tissue/fragments) during implantation (during insertion of the implant into the patient's body). These omissions may have a size of 1.0 to 1.5 mm, 1.5 to 2.0 mm, 2.0 to 2.5 mm or 2.5 to 2.8 mm. If larger bone fragments are to be inserted into the implant or if the implant is to accommodate larger bone fragments, the implant can accordingly have fixation structures with enlarged geometry to accommodate the individual bone fragments.

The materials used for the implant according to the invention are available in powder form, granular form, and as a liquid or viscous mixture, wherein the materials are mixed together in different amounts and compositions of substances. The granular form is particularly important here, since the desired geometric and biological properties of the implant are controlled by the energy input and this is dependent on the powder form and granular form.

Spherical particles with sizes of 5-18 μm and 25-120 μm may be used, wherein the metallic components are smaller than the ceramic particles. Furthermore, the ceramic particles may have fully or partially cubic shapes with edge lengths of 5-25 μm as well as of 40-60 μm. In addition, the first components of the ceramic particles may include a mixture of geometrically non-uniform powder particles and the ceramic component may have a crystalline or partially crystalline arrangement.

The implant according to the invention, which is structurally built up in layers with gradual or stepwise degradation (degradability), enables cell-type-specific ingrowth of the implant into the patient's body with regard to cell migration (active change of location of cells or cell assemblies in the patient's tissue). Furthermore, such an implant advantageously causes a defined activation of cell physiological processes at and in the implant.

In the following, an embodiment of the implant according to the invention as well as the method for manufacturing the implant are described in detail with reference to the attached drawings.

The following is shown:

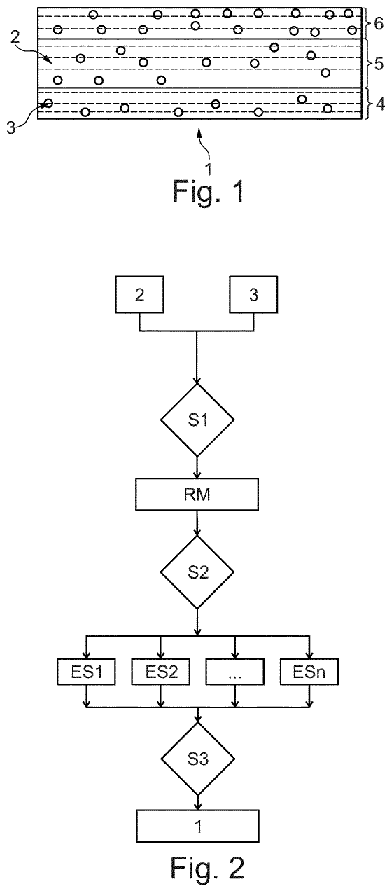

FIG. 1 shows a cross-sectional view of the implant according to the invention; and

FIG. 2 shows a flow diagram depicting the steps for manufacturing an implant.

The figures are merely schematic in nature and are intended only for the purpose of understanding the invention. The embodiment is purely exemplary.

FIG. 1 shows the implant 1, which has the carrier material 2 and a donor material 3. At the same time, the layer structure of the implant 1 can be seen. The layers are arranged in such a way that, in this configuration example, the first layers 4 are arranged at the bottom, the middle layers 5 are located above the first layers 4, and the last layers 6 are arranged at the top (above the middle layers 5) in this view. The first, middle and last layers 4, 5, 6 have different densities/porosities.

FIG. 2 shows a flow chart which illustrates the individual steps for obtaining the implant 1 according to the invention. In a first step S1, the ceramic carrier material 2 and the donor material 3, which contains resorbable components, are mixed together to form a raw mixture RM. In the subsequent step S2, the individual components of this raw mixture RM are bonded to each other in a spatially-resolved manner by laser sintering, so that a plurality of individual layers, e.g. a first individual layer ES1, a second individual layer ES2 and further individual layers are produced, wherein any individual layer is characterized by ESn. In the third step S3, which follows on step S2, these individual layers ES1, ES2, . . . , ESn are superimposed and bonded to each other under energy input so that the finished implant 1 is obtained as a product.

### LIST OF REFERENCE SIGNS

1 implant
2 carrier material
3 donor material
4 first layers
5 middle layers
6 last layers
ES1 first individual layer
ES2 second individual layer
ESn n^{th} (any) individual layer
RM raw mixture
S1 first step
S2 second step
S3 third step

1. Implant for insertion into a patient, having an at least partially resorbable and at least in partial regions porous implant body made of a ceramic carrier material, which is provided with a donor material that, in the implanted state, emits ions for influencing the patient's cellular metabolism, wherein the donor material intersperses the carrier material so that the donor material is present throughout the entire implant volume,
wherein
the implant comprises first layers, last layers and middle layers, which are surrounded by the first and last layers, wherein the first, last and middle layers have different densities/porosities.

2. Implant according to claim 1, wherein the donor material comprises ceramic particles and/or metallic particles.

3. Implant according to claim 1, wherein the implant body is divided into layers or into partial regions of different density and/or porosity.

4. Implant according to claim 1, wherein individual pores in the implant body are connected to each other via connection channels.

5. Implant according to claim 4, wherein the donor material is arranged and concentrated in the carrier material in such a way that, when the ions are released in the implanted state, the connection channels necessarily result, or that the connection channels and implant body are already present before insertion into the patient.

6. Implant according to claim 1, wherein the implant body has a total porosity between 3% and 60%.

7. Implant according to claim 1, wherein the pore size of the pores in the implant body lies in a range of 300 μm to 1,500 μm.

8. Implant according to claim 1, wherein the ceramic carrier material is provided in the form of powder or granular ceramic particles.

9. Implant according to claim 2, wherein the ceramic particles and the metal particles are spherical with a particle size between 5-18 μm for the metal particles and between 25-120 μm for the ceramic particles and/or cubic with an edge length between 5-25 μm for the metal particles and between 40-60 μm for the ceramic particles.

10. A method of manufacturing an implant according to claim 1, comprising the steps:
a) mixing of carrier material and donor material into a raw mixture,
b) spatially-resolved bonding of the raw mixture (RM) into a plurality of individual layers (ES1, ES2, ESn), and
c) superimposing and layer-by-layer bonding of the plurality of individual layers to form the finished implant body.
